(19) United States
(12) Patent Application Publication
(10) Pub. No.: US 2015/0066008 A1
(43) Pub. Date: Mar. 5, 2015

(54) DEVICE FOR AUTOMATIC ANCHOR UNDEPLOYMENT AND RETRACTION

(71) Applicant: Calore Medical Ltd., Or Akiva (IL)

(72) Inventors: David Maier Neustadter, Nof Ayalon (IL); Giora Kornblau, Binyamina (IL)

(73) Assignee: Calore Medical Ltd.

(21) Appl. No.: 14/471,435

(22) Filed: Aug. 28, 2014

Related U.S. Application Data
(60) Provisional application No. 61/937,543, filed on Feb. 9, 2014.
(60) Provisional application No. 61/872,723, filed on Sep. 1, 2013.
(60) Provisional application No. 62/015,968, filed on Jun. 23, 2014.

(51) Int. Cl. A61B 17/12; A61B 18/04

(52) U.S. Cl. CPC A61B 17/12109; A61B 18/04; A61B 2017/1209; A61B 2017/12054; A61B 2018/0063
USPC 606/28

(57) ABSTRACT

A tissue closure device and a treatment method for closing an opening in a tissue wall are described. The tissue closure device comprises a heating element and a removable anchor having an anchor shaft and an occluding element at or near a distal end of the anchor shaft. The occluding element of the anchor is configured to be expanded and collapsed. The treatment method comprises introducing the occluding element of the anchor into a body canal through an opening in a tissue wall of the body canal, and deploying the anchor such that the occluding element expands to temporarily occlude the opening in the tissue wall. The tissue region surrounding the opening is then heated to cause shrinkage of collagen-containing tissue, thereby contracting the opening in the tissue wall. When a predefined trigger associated with the treatment method is met, the anchor is automatically undeployed and retracted from the body canal.

### CROSS-REFERENCES TO RELATED APPLICATIONS

This application claims priority to U.S. Provisional Application No. 61/872,723, filed Sep. 1, 2013, U.S. Provisional Application No. 61/937,543, filed Feb. 9, 2014, and U.S. Provisional Application No. 62/015,968, filed Jun. 23, 2014, all of which are incorporated herein by reference in their entirety.

### TECHNICAL FIELD

The present disclosure relates generally to a tissue closure device, and more specifically, to a device for automatic undeployment and retraction of a removable anchor following treatment of an opening in a tissue wall.

### BACKGROUND

Heat may be used to shrink tissue containing collagen in various surgical and diagnostic procedures when treating a patient. For example, heat may be used to close and/or heal perforations, openings, or punctures in the patient's tissue walls. In particular, heat may be used to close arteriotomies on blood vessel walls by denaturing collagen within the tissue, thereby shrinking the tissue around the puncture, and/or by inducing blood coagulation. In such procedures, a physician may introduce an anchor device into the patient's blood vessel to temporarily occlude the puncture and provide hemostasis during the closure procedure. The physician may then introduce a tissue closure device into the body to cause shrinkage of the collagenous tissue in the blood vessel wall and/or cause blood coagulation around the puncture site. Finally, the physician may remove the anchor device to complete closure of the puncture.

The timing of the anchor removal relative to the tissue shrinkage and/or blood coagulation process, as ell as the smoothness of the removal motion, can be critical to the success of the closure procedure. Early removal may result in bleeding that will interfere with the tissue heating and/or blood coagulation process, and late removal may result in damage to the newly formed tissue seal at the puncture site, which may lead to reopening of the repaired/closed puncture. Therefore, devices and methods that prevent these potential adverse consequences are desirable.

### SUMMARY

The present disclosure is directed to a tissue closure device that allows automatic undeployment and retraction of an anchor element when a predefined trigger associated with a closure procedure is met. Some aspects of the present disclosure include a tissue closure device comprising a treatment applicator and an anchor element for temporarily occluding an opening in a tissue wall while the treatment applicator is used to close or heal the opening. One application of the tissue closure device and treatment method of the present disclosure is the thermal closure of puncture sites (i.e., arteriotomies) on blood vessel walls. The application of the device and method of the present disclosure are not limited to the blood vasculature, and may be applied to any vessel, duct, canal, tubular structure, and/or cavity in the body. It is to be understood that the term “body canal” in this disclosure refers to any blood vessel, duct, canal, tubular structure, and/or tissue tract within the body.

In one embodiment, a tissue closure device for treating an opening in a tissue wall is disclosed. The tissue closure device comprises a removable anchor having an anchor shaft and an occluding element at or near a distal end of the anchor shaft. The occluding element is configured to be deployed within a body canal for temporarily closing an opening in a tissue wall of the body canal. The tissue closure device further comprises a housing configured to be located external to a patient when the occluding element is located within the body canal, a treatment applicator for applying a tissue closure treatment to the opening on the tissue wall, and a controller positioned in the housing and configured to automatically cause the removable anchor to be undeployed from the body canal when a predetermined trigger is met.

In another embodiment, a method of closing an opening in a tissue wall of a body canal is disclosed. The method comprises providing a removable anchor comprising an anchor shaft and an occluding element at or near a distal end of the anchor shaft. The occluding element is expandable and collapsible within the body canal. The method further comprises introducing the occluding element into the body canal through the opening in the tissue wall and deploying the anchor thereby causing the occluding element to expand within the body canal. The method further comprises applying a tissue closure treatment to close the opening in the tissue wall, monitoring a treatment parameter associated with the tissue closure treatment, and automatically undeploying the anchor when a predetermined trigger associated with the treatment parameter is met.

Other embodiments of this disclosure are contained in the accompanying drawings, description, and claims. Thus, this summary is exemplary only, and is not to be considered restrictive.

### BRIEF DESCRIPTION OF DRAWINGS

The accompanying drawings, which are incorporated in and constitute a part of this specification, illustrate the disclosed embodiments and together with the description, serve to explain the principles of the various aspects of the disclosed embodiments. In the drawings:

FIG. 1 illustrates an automated undeployment and retraction mechanism of a tissue closure device, in accordance with exemplary embodiments of the present disclosure; and

FIG. 2 is a flow diagram of the steps of an exemplary vascular closure procedure, in accordance with exemplary embodiments of the present disclosure.

It is to be understood that both the foregoing general description and the following detailed description are exemplary and explanatory only and are not restrictive of the disclosed embodiments, as claimed.

### DESCRIPTION OF EXEMPLARY EMBODIMENTS

Reference will now be made to certain embodiments consistent with the present disclosure, examples of which are illustrated in the accompanying drawings. Wherever possible, the same reference numbers are used throughout the drawings to refer to the same or like parts.

The present disclosure describes a tissue closure device comprising an anchor and a treatment applicator for closing or healing an opening in a tissue wall of a body canal. An anchor of the present disclosure is configured to be removable from within the body canal where it is deployed during the treatment of the opening in the tissue wall. The disclosed tissue closure device and treatment method may be employed to close or heal perforations, openings, or punctures in any tissue region of a patient's body.

Exemplary embodiments of an anchor of the present disclosure comprise an anchor shaft and an occluding element at or near a distal end of the anchor shaft. The occluding element of the anchor is configured to be expanded and collapsed within the body canal, for example, within a blood vessel. In exemplary embodiments, the occluding element of the anchor is inserted into the body canal at the beginning of a tissue closure procedure, through the opening which is to be closed, and removed during or after the closure procedure. The anchor may perform one or more of the following functions when inserted into the body canal: 1) provide an indication of the depth of the body canal from the exterior of the body surface; 2) provide mechanical stabilization of the body canal to aid in the insertion of the treatment applicator; 3) provide guidance as to the location of the opening or perforation in the tissue wall of the body canal; and 4) provide temporary hemostasis during part or all of the closure procedure.

After the anchor is inserted into the body canal, it is deployed, which results in expansion, opening, or change in the geometrical shape of the occluding element such that it can no longer pass through the opening in the tissue wall. The expansion of the occluding element may also provide temporary hemostasis. The treatment applicator of the tissue closure device is then activated to close the puncture in the tissue wall When a predefined trigger associated with the closure procedure is met, the anchor is undeployed, which results in closing or collapsing of the occluding element such that it can pass through the opening in the tissue wall. In exemplary embodiments, the predefined trigger is a treatment parameter associated with the closure procedure. In one such embodiment, the predefined trigger is an amount of time after the treatment applicator has been activated. In other embodiments, the predefined trigger is a sensed parameter, for example, the temperature of the surrounding tissue region or the temperature of the treatment applicator. In yet other embodiments, the predefined trigger is an amount of time after a sensed parameter, for example, the temperature of the surrounding tissue region or the temperature of the treatment applicator, has reached a predetermined level.

In some embodiments, undeployment of the anchor device is automated, i.e., once the predefined trigger is met, the anchor device is automatically undeployed. In other embodiments, undeployment is done manually by the user when the predefined trigger is met. In yet other embodiments, the occluding element of the anchor has a natural tendency to return to its undeployed configuration. Once the anchor device is undeployed, it is retracted from the body canal, i.e., the anchor device is removed from the inside of the body canal.

Exemplary embodiments of the treatment applicator may include a heating element configured to deliver heat to a tissue region containing collagen. The heating element may include any electrical, chemical, mechanical, or other mechanism for causing heat, such as, for example, ultrasound heating, RF heating, laser heating, microwave heating, or any device capable of converting electricity into heat through a process of resistive or Joule heating. The heating element may be encased in a heat-conductive housing or may be otherwise provided to enable the resulting heat to be directed to a target tissue area. Exemplary embodiments may also include a support for positioning the heating element at a location to effect delivery of heat to the tissue region. By way of example, the support may be an elongate element. The particular shape and construction of an elongated support may vary, and might include, by way of example only, one or more of a tube, rod, shaft, bar, rib, or column.

In exemplary embodiments, the elongate element as well as the heat-conductive housing may include a lumen with an opening therethrough allowing for placement of the treatment applicator over the anchor shaft. Further, in exemplary embodiments, a control system may be housed within a handle of the treatment applicator. In some such embodiments, the control system may include a power source, which may be housed within the handle as part of the control system itself, or as a separate component. The power source may be configured for electrical connection to the heating element. In exemplary embodiments, the power source may also be electrically connected to a temperature sensor associated with, or provided in close proximity to, the heating element. The temperature of the target tissue region, or the temperature of the heating element may be monitored by the temperature sensor, and the power delivered to the heating element may be adjusted to maintain a preselected thermal profile. A microprocessor or a controller may be included and incorporated into control system to modify the power delivered to heating element based on the temperature information received by the controller or the microprocessor from temperature sensor. The temperature sensor itself may include any structure capable of either detecting or measuring temperature, including, for example, a thermometer, bimetal, thermocouple, resistance thermometer, silicon bandgap temperature sensor, or any other arrangement or structure capable of providing feedback indicative of temperature.

In exemplary embodiments that use heat to close an opening in the tissue wall, the tissue closure procedure comprises introducing the occluding element of the anchor into the body canal through the opening that is to be closed and deploying the anchor such that the occluding element expands to temporarily occlude the opening. The tissue region surrounding the opening is then heated with the treatment applicator, i.e., the heating element, to cause shrinkage of collagen-containing tissue and/or cause blood coagulation, thereby closing the opening. When a predefined trigger associated with the closure procedure is met, the anchor is undeployed. In such embodiments, temperature of the target tissue region, and/or the temperature of a distal region of the heating element, may be monitored while heat is applied to the tissue. In one such embodiment, the predefined trigger is a temperature of the target tissue region and/or the temperature of the heating element. In another embodiment, the predefined trigger is an amount of time after the target tissue region and/or the temperature of the heating element reaches a predetermined temperature. Further, in some embodiments, the anchor may be undeployed while the heating element is being energized. In some other embodiment, the anchor may be undeployed while the tissue region is being heated. Some embodiments of the present disclosure also include an automated retraction mechanism for retracting or removing of the occluding element during or after the undeployment of the anchor.

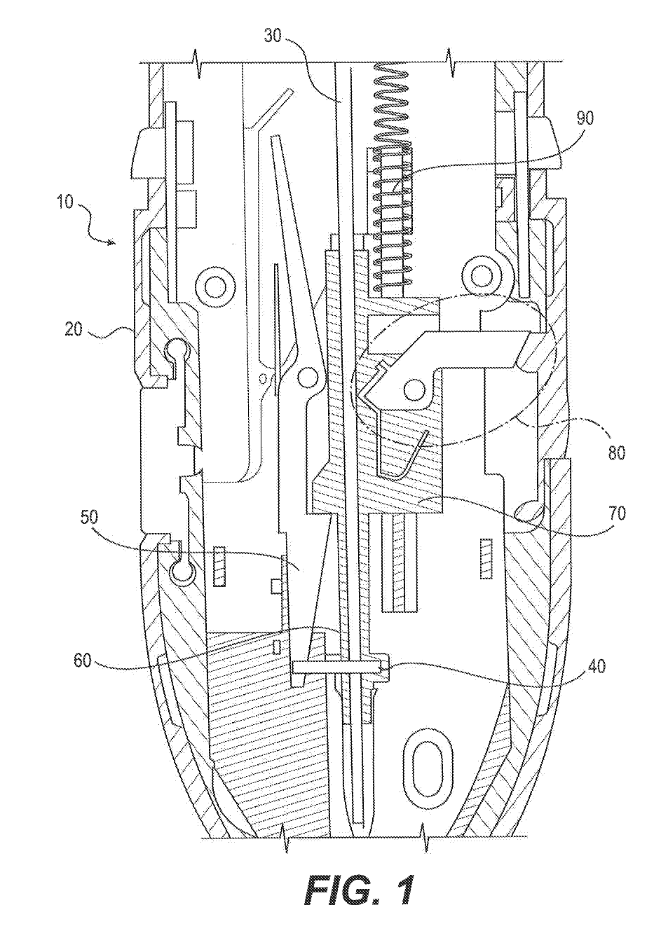

FIG. 1 shows an exemplary embodiment of an automated anchor undeployment and retraction mechanism of a tissue closure device 10. The automated anchor undeployment and retraction mechanism of tissue closure device 10 allows a user to automate the timing of the undeployment and/or retraction, which may provide more consistency and precision in undeployment and/or retraction than that achieved by manual actuation. In exemplary embodiments, the anchor undeployment and retraction mechanism is positioned in a housing 20 of tissue closure device 10. An anchor shaft 30 of the removable anchor passes through housing 20 in close proximity to the anchor undeployment and/or retraction mechanism positioned therein.

In one exemplary embodiment, the anchor undeployment mechanism may include a means for releasing a locking mechanism within the anchor shaft (that locks the anchor in the deployed configuration), thereby allowing the anchor to return to its undeployed configuration. Means for releasing a locking mechanism may include, for example, rotating, pushing, pulling, bending, or compressing a mechanical element in order to release it from its locked configuration. In another exemplary embodiment, the anchor undeployment mechanism may include a means for releasing pressure from a tube within the anchor shaft (that locks the anchor in the deployed configuration), thereby allowing the anchor to return to its undeployed configuration. Means for releasing pressure from a tube may include, for example, cutting or puncturing the tube, releasing a valve on the tube, or increasing the volume of the tube by moving a plunger.

In yet another exemplary embodiment, illustrated in FIG. 1 the anchor undeployment mechanism may include a means for cutting or severing an actuator element (not shown) provided within the anchor shaft. The actuator element may hold the anchor in its deployed configuration, and cutting, severing, or loosening of the actuator element may allow the anchor to return to its undeployed configuration. In exemplary embodiments, the actuator element is a taut wire that holds the anchor in the deployed configuration.

In some embodiments, the means for cutting the actuator element may include cutting with a sharp blade. In such embodiments, the anchor undeployment mechanism may include a spring activated or motor activated mechanism that, when actuated, presses the blade against the anchor shaft with sufficient force to cut through the anchor shaft and sever the actuator element running through the anchor shaft.

In other embodiments, the means for cutting the actuator element may include cutting with heat. In some such embodiments, cutting with heat may include a heating element, e.g., an electrically-heated resistive metal wire 40, as shown in FIG. 1. In some embodiments that use a taut wire as an actuator element, electrically-heated resistive metal wire 40 may soften the taut wire such that it stretches and is no longer taut. In other embodiments, cutting with heat may include melting the actuator element with electrically-heated resistive metal wire 40. In exemplary embodiments, the anchor undeployment mechanism may include electrically-heated resistive metal wire 40 mounted on a spring-loaded support 50 such that electrically-heated metal wire 40 presses against anchor shaft 30. When electrically-heated metal wire 40 is actuated, either manually or by a predefined trigger, it melts through anchor shaft 30 and severs the actuator element within anchor shaft 30.

In exemplary embodiments, the anchor undeployment mechanism may include a separator material 60 positioned around the anchor shaft, on or around which electrically-heated resistive metal wire 40 initially rests. In such embodiments, when the electrically-heated resistive metal wire 40 is activated, it melts through separator material 60 and then melts anchor shaft 30 and severs the actuator element. In some embodiments, separator material 60 may be a sleeve, sheath, or a tube through which anchor shaft 30 passes and around which electrically-heated resistive metal wire 40 is positioned. In other embodiments, separator material 60 may be in the form of a hook or pin which initially restrains the electrically-heated resistive metal wire 40 and does not allow it to contact anchor shaft 30 until separator material 60 has been melted through.

The initiation of the undeployment mechanism may be controlled by an electronic controller or processor provided in a handle or housing 20 of tissue closure device 10. In some embodiments, electronic controller or processor for the undeployment mechanism may be the same as the controller used for modifying the power delivered to the heating element of the treatment applicator (for heating the tissue containing collagen). In other embodiments, the handle or housing 20 of tissue closure device 10 may comprise a controller or a microprocessor dedicated to the undeployment mechanism. The undeployment may be initiated at a predefined time after one or more events of the tissue closure procedure. For example, the undeployment may be initiated during the tissue closure procedure, or after the tissue closure procedure is complete. In some exemplary embodiments, the undeployment may be initiated at a predefined time after the activation of the heating element of the treatment applicator. In other embodiments, the undeployment may be initiated at a predefined time after the heating element of the treatment applicator, or the surrounding tissue, reaches a certain temperature (e.g., the heating element of the treatment applicator reaches a temperature of 90° C., or the surrounding tissue reaches a temperature of 70° C.). In such embodiments, the controller of the undeployment mechanism may be operatively coupled to the temperature sensor of the treatment applicator so that the undeployment mechanism can be operatively tied to the temperature of a heating element of the treatment applicator, or the temperature of the surrounding tissue.

Some embodiments of the present disclosure may also include an automated retraction mechanism for retraction or removal of the occluding element during or after the undeployment of the anchor. In some embodiments, the occluding element may be retracted while the heating element is being energized. In some other embodiment, the occluding element may be retracted while the tissue region is being heated.

In exemplary embodiments, as illustrated in FIG. 1, the anchor retraction mechanism may include a carriage 70 and a grasping element 80 connected to carriage 70. Carriage 70 and grasping element 80 may be configured to grasp anchor shaft 30 during or after the undeployment of the anchor. In some embodiments, carriage 70 may be loaded with a spring 90 and held in place by an activation mechanism which, when released, actuates carriage 70. In some embodiments, the anchor undeployment mechanism may serve as an activation mechanism that releases spring 90 and actuates carriage 70. In one such embodiment, spring-loaded support 50 of the undeployment mechanism may act as the activation mechanism for carriage 70.

When actuated, carnage 70 may cause grasping element 80 to grasp anchor shaft 30. In exemplary embodiments, grasping element 80 may be a ratchet mechanism, which allows anchor shaft 30 to move in one direction, but resists movement of anchor shaft 30 in the opposite direction.

Alternatively, in some exemplary embodiments, the automated anchor retraction mechanism may comprise a motor and a shaft grasping element. When the motor is activated, the shaft grasper is actuated to move in such a way as to grasp and retract the anchor.

As with automated undeployment mechanism, the initiation of the automated retraction mechanism may be controlled by an electronic controller or processor provided in a handle or housing 20 of tissue closure device 10. In some embodiments, the electronic controller or processor for the retraction mechanism may be the same as the controller or processor used for the undeployment mechanism. In some embodiments, the controller for initiating the undeployment and/or retraction mechanism may be the same as the controller or processor for modifying the power delivered to the heating element of the treatment applicator (for heating the tissue containing collagen). The retraction may be initiated at a predefined time after one or more events of the tissue closure procedure. For example, the retraction may be initiated during the tissue closure procedure, or after the tissue closure procedure is complete. In some exemplary embodiments, the retraction may be initiated immediately following the undeployment. In other embodiments, the retraction may be initiated at a predefined time after the activation of the heating element of the treatment applicator, or at a predefined time after the heating element of the treatment applicator, or the surrounding tissue, reaches a certain temperature.

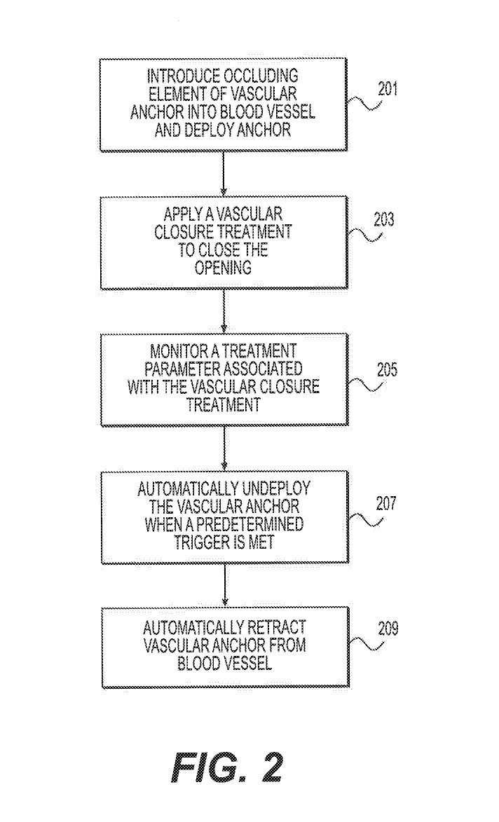

FIG. 2 illustrates the steps of an exemplary vascular closure procedure using an exemplary vascular anchor comprising an occluding element that is expandable and collapsible within a blood vessel. The closure procedure described here, with reference to FIG. 2, may be used to close or heal perforations, openings, or punctures in any tissue region of a patient's body. As shown in FIG. 2, a first step (step 201) of the treatment procedure is introducing the occluding element into the blood vessel through an opening in the blood vessel wall and deploying the anchor thereby causing the occluding element to expand within the blood vessel. The next step (step 203) is applying a vascular closure treatment to close the opening (e.g., an arteriotomy) on the blood vessel wall. In one exemplary embodiment, the vascular closure treatment comprises applying heat to the tissue region surrounding the opening to cause shrinkage of collagen containing tissue and/or cause blood coagulation. The next step (step 205) is monitoring a treatment parameter (e.g., temperature of the tissue region, temperature of the treatment applicator, or the time that has passed since some treatment related event) associated with the vascular closure treatment. When a predetermined trigger associated with the treatment parameter is met, the vascular anchor is automatically undeployed (step 207). Following undeployment, the vascular anchor is automatically retracted from the blood vessel (step 209).

In exemplary embodiments, tissue closure device 10 may also provide indications of the status of the occluding element, i.e., deployed, undeployed, and retracted. Further, in some embodiments, tissue closure device 10 may also provide indications of the status of the treatment procedure, i.e., started, need to perform action, ended, etc.

The foregoing description has been presented for purposes of illustration. It is not exhaustive and is not limited to the precise forms or embodiments disclosed. Modifications and adaptations will be apparent to those skilled in the art from consideration of the specification and practice of the disclosed embodiments.

Moreover, while illustrative embodiments have been described herein, the scope of any and all embodiments include equivalent elements, modifications, omissions, combinations (e.g., of aspects across various embodiments), adaptations and/or alterations as would be appreciated by those skilled in the art based on the present disclosure. The limitations in the claims are to be interpreted broadly based on the language employed in the claims and not limited to examples described in the present specification or during the prosecution of the application. The examples are to be construed as non-exclusive. Furthermore, the steps of the disclosed methods may be modified in any manner, including by reordering steps and/or inserting or deleting steps. It is intended, therefore, that the specification and examples be considered as illustrative only, with a true scope and spirit being indicated by the following claims and their full scope of equivalents.

1. A tissue closure device, comprising:
a removable anchor comprising an anchor shaft and an occluding element at or near a distal end of the anchor shaft, wherein the occluding element is configured to be deployed within a body canal and located at an opening in a tissue wall of the body canal;
a housing configured to be located external to a patient when the occluding element is located within the body canal;
a treatment applicator for applying a tissue closure treatment to the opening on the tissue wall; and
a controller positioned in the housing and configured to automatically cause the removable anchor to be undeployed when a predetermined trigger is met.

2. The device of claim 1, wherein the treatment applicator comprises a heating element configured to provide controlled heat to a tissue region surrounding the opening in the tissue wall to cause shrinkage of tissue containing collagen.

3. The device of claim 2, wherein the predetermined trigger is an amount of time after the heating element reaches a predefined temperature.

4. The device of claim 1, wherein the predetermined trigger is a predefined amount of time after the treatment applicator is activated.

5. The device of claim 1, wherein the occluding element is expandable and collapsible within the blood vessel.

6. The device of claim 5, wherein the occluding element is maintained in an expanded configuration within the body canal by an actuator element provided within at least a portion of the anchor shaft.

7. The device of claim 6, wherein the actuator element is a taut wire which maintains the occluding element in its expanded configuration.

8. The device of claim 6, further comprising a severing element within the housing.

9. The device of claim 8, wherein the severing element is configured to sever the actuator element when the predetermined trigger is met thereby causing the anchor to be undeployed.

10. The device of claim 8, wherein the severing element is a blade.

11. The device of claim 8, wherein the severing element includes a heating component configured to apply heat to the wire causing the wire to melt.

12. The device of claim 11, wherein the heating component is a resistively-heated metal wire.

13. The device of claim 11, wherein the anchor shaft is encased in a sheath in an area of the heating component, and wherein the heating component is configured to melt through the sheath and the anchor shaft before heating the actuator element.

14. The device of claim 1, further comprising a sensor associated with the treatment applicator for detecting at least one treatment parameter.

15. The device of claim 14, wherein the controller is configured to access information related to the at least one treatment parameter from the sensor and to automatically cause the anchor to be undeployed when the predetermined trigger associated with the at least one treatment parameter is met.

16. The device of claim 1, further comprising an automated retraction mechanism comprising a grasping element configured to grasp the anchor shaft during or after undeployment of the anchor.

17. The device of claim 16, wherein the automated retraction mechanism further comprises a spring-loaded carriage associated with the grasping element.

18. The device of claim 16, wherein the automated retraction mechanism further comprises a motor associated with the grasping element.

19. A method of closing an opening in a tissue wall of a body canal, comprising:
providing a removable anchor comprising an anchor shaft and an occluding element at or near a distal end of the anchor shaft, wherein the occluding element is expandable and collapsible within the body canal;
introducing the occluding element into the body canal through the opening in the tissue wall and deploying the anchor thereby causing the occluding element to expand within the body canal;
applying a tissue closure treatment to the opening in the tissue wall;
monitoring a treatment parameter associated with the tissue closure treatment; and
automatically undeploying the anchor hen a predetermined trigger associated with the treatment parameter is met.

20. The method of claim 19, wherein applying the tissue closure treatment to the opening in the tissue wall comprises applying heat to a tissue region surrounding the opening to cause shrinkage of collagen-containing tissue.

21. The method of claim 20, wherein applying the tissue closure treatment to the opening in the tissue wall comprises introducing a heating device percutaneously over the anchor shaft to a location proximate the opening in the tissue wall.

22. The method of claim 21, wherein monitoring a treatment parameter comprises monitoring a temperature of at least one of the heating device and the tissue region surrounding the opening.

23. The method of claim 21, wherein the step of automatically undeploying the anchor is performed while the heating device is energized.

24. The method of claim 21, wherein the step of automatically undeploying the anchor is performed while the heating device is heating the tissue region surrounding the opening.

25. The method of claim 21, further comprising retracting the anchor from the body canal.

26. The method of claim 25, wherein the anchor is automatically retracted after the anchor is undeployed.

27. The method of claim 25, wherein the anchor is automatically retracted during the undeployment of the anchor.

28. The method of claim 27, wherein the anchor is automatically retracted while the heating device is energized.

29. The method of claim 27, wherein the anchor is automatically retracted while the heating device is heating the tissue region surrounding the opening.

30. The method of claim 19, wherein automatically undeploying the anchor comprises severing an actuator element which maintains the occluding element in its expanded configuration.
